(19) United States
(12) Patent Application Publication
(10) Pub. No.: US 2026/0283580 A1
(43) Pub. Date: Sep. 24, 2026

(54) MEDICAL IMAGE PROCESSING APPARATUS, X-RAY DIAGNOSTIC APPARATUS, AND STORAGE MEDIUM

(71) Applicant: CANON MEDICAL SYSTEMS CORPORATION, Tochigi (JP)

(72) Inventor: Haruki IWAI, Otawara (JP)

(73) Assignee: CANON MEDICAL SYSTEMS CORPORATION, Tochigi (JP)

(21) Appl. No.: 19/561,843

(22) Filed: Mar. 10, 2026

(30) Foreign Application Priority Data
Mar. 18, 2025 (JP) 2025-043902
Jan. 26, 2026 (JP) 2026-010466

(51) Int. Cl. A61B 6/00; G06T 3/60; G06T 11/60

(52) U.S. Cl. CPC A61B 6/5247; G06T 3/60; G06T 11/60; G06T 2210/41

(57) ABSTRACT

A medical image processing apparatus according to an embodiment includes a processing circuitry that acquires an X-ray image and an optical image of a subject and that superimposes first identification information including a figure on the optical image and superimposes second identification information including a figure according to the difference in the line-of-sight direction from the optical image on the X-ray image.

### CROSS-REFERENCE TO RELATED APPLICATIONS

This application is based upon and claims the benefit of priority from Japanese Patent Application No. 2025-043902, filed on Mar. 18, 2025; and Japanese Patent Application No. 2026-010466, filed on Jan. 26, 2026, the entire contents of which are incorporated herein by reference.

### FIELD

Embodiments described herein relate generally to a medical image processing apparatus, an X-ray diagnostic apparatus, and a storage medium.

### BACKGROUND

During an examination using an X-ray diagnostic apparatus, optical images may be acquired in addition to X-ray images. Various kinds of information can be obtained from the optical images, including the state of a subject when the X-ray images are acquired or the surrounding conditions including doctors and an examination room. The optical image is useful information for diagnosis in itself and can also be used as additional information to use the X-ray image. However, the appearances of the X-ray image and the optical image do not always match, and the correspondence between them may be difficult to understand.

### BRIEF DESCRIPTION OF THE DRAWINGS

FIG. 1 is a block diagram of an example of a medical image processing system according to a first embodiment;

FIG. 2 is a block diagram of an example of the configuration of an X-ray diagnostic apparatus according to the first embodiment;

FIG. 3 is a schematic of an example of an imaging unit according to the first embodiment;

FIG. 4A is a schematic of an example of an optical image according to the first embodiment;

FIG. 4B is a schematic of an example of an X-ray image according to the first embodiment;

FIG. 5A is a schematic of a display example of identification information according to the first embodiment;

FIG. 5B is a schematic of a display example of the identification information according to the first embodiment;

FIG. 6A is a schematic of a display example of the identification information according to a second embodiment;

FIG. 6B is a schematic of a display example of the identification information according to the second embodiment;

FIG. 6C is a schematic of a display example of the identification information according to the second embodiment;

FIG. 7A is a schematic of a display example of the identification information according to the second embodiment;

FIG. 7B is a schematic of a display example of the identification information according to the second embodiment; and

FIG. 8 is a schematic of a display example of the identification information according to a third embodiment.

### DETAILED DESCRIPTION

A medical image processing apparatus according to an embodiment includes processing circuitry that acquires an X-ray image and an optical image of a subject and that superimposes first identification information including a figure on the optical image and superimposes second identification information including a figure according to difference in a line-of-sight direction from the optical image on the X-ray image.

An X-ray diagnostic apparatus according to an embodiment includes processing circuitry that acquires an X-ray image of a subject and that superimposes first identification information including a figure on an optical image of the subject and superimposes second identification information including a figure according to difference in a line-of-sight direction from the optical image on the X-ray image.

A non-transitory computer readable medium according to an embodiment includes instructions that cause a computer to execute acquiring an X-ray image and an optical image of a subject, and superimposing first identification information including a figure on the optical image and superimposing second identification information including a figure according to difference in a line-of-sight direction from the optical image on the X-ray image.

Exemplary embodiments of a medical image processing apparatus, an X-ray diagnostic apparatus, and a computer program are described below in greater detail with reference to the accompanying drawings.

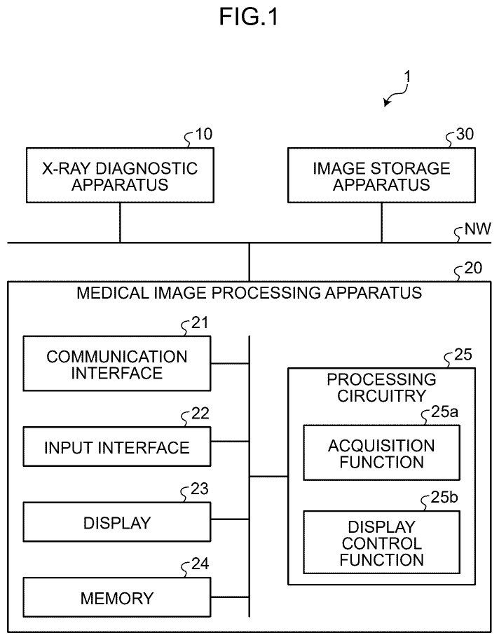

A first embodiment describes a medical image processing system 1 illustrated in FIG. 1 as an example. The medical image processing system 1 includes an X-ray diagnostic apparatus 10, a medical image processing apparatus 20, and an image storage apparatus 30. The X-ray diagnostic apparatus 10, the medical image processing apparatus 20, and the image storage apparatus 30 are communicably connected via a network NW.

The X-ray diagnostic apparatus 10 is an apparatus that acquires X-ray images. The X-ray diagnostic apparatus 10 according to the present embodiment includes an optical camera and acquires optical images besides the X-ray images. The X-ray diagnostic apparatus 10 will be described later in greater detail.

The image storage apparatus 30 is an apparatus that stores therein the X-ray images and the optical images acquired by the X-ray diagnostic apparatus 10. While one X-ray diagnostic apparatus 10 alone is illustrated in FIG. 1, the image storage apparatus 30 may be connected to a plurality of X-ray diagnostic apparatuses 10. The image storage apparatus 30 may further store therein medical images acquired by another type of medical image diagnostic apparatus (modality) different from the X-ray diagnostic apparatus. The image storage apparatus 30 is, for example, a picture archiving and communication system (PACS) server.

The medical image processing apparatus 20 is an apparatus that displays X-ray images and optical images. Specifically, the medical image processing apparatus 20 displays the X-ray image and the optical image such that the correspondence between them can be readily grasped. As illustrated in FIG. 1, the medical image processing apparatus 20 includes, for example, a communication interface 21, an input interface 22, a display 23, a memory 24, and processing circuitry 25.

The communication interface 21 controls transmission and communications of various data transmitted to and received from other apparatuses or systems connected to the medical image processing apparatus 20 via the network NW. Specifically, the communication interface 21 is connected to the processing circuitry 25 and outputs data received from other apparatuses or systems to the processing circuitry 25 or transmits data output from the processing circuitry 25 to other apparatuses or systems. The communication interface 21 is implemented by, for example, a network card, a network adapter, or a network interface controller (NIC).

The input interface 22 receives various input operations from an operator of the medical image processing apparatus 20, converts the received input operation into electrical signals, and outputs them to the processing circuitry 25. The input interface 22 is implemented by, for example, a mouse, a keyboard, a trackball, a switch, a button, a joystick, a touchpad on which the operator performs an input operation by touching an operation surface, a touchscreen that integrates a display screen and a touchpad, a non-contact input circuit with an optical sensor, or a voice input circuit. The input interface 22 may be configured as a tablet terminal device or the like that can wirelessly communicate with the medical image processing apparatus 20 itself. The input interface 22 may be a circuit that receives the input operation from the operator by motion capture. For example, the input interface 22 can receive the operator's body movement, line of sight, or the like as the input operation by processing signals acquired via a tracker or images acquired about the operator. The input interface 22 is not limited only to those with physical operation components, such as a mouse and a keyboard. Examples of the input interface 22 also include an electrical signal processing circuit that receives electrical signals corresponding to an input operation from an external input device provided separately from the medical image processing apparatus 20 and outputs the electrical signals to the processing circuitry 25.

The display 23 displays various kinds of information. For example, the display 23 displays a graphical user interface (GUI) to receive various instructions and settings from the operator via the input interface 22. The display 23 displays the X-ray images and the optical images acquired by the X-ray diagnostic apparatus 10. The display 23 is, for example, a liquid crystal display or a cathode ray tube (CRT) display. The display 23 may be a desktop display or may be configured as a tablet terminal device or the like that can wirelessly communicate with the medical image processing apparatus 20 itself.

The medical image processing apparatus 20 may include a projector instead of or in addition to the display 23. The projector can project images on a screen, wall, floor, or the like under the control of the processing circuitry 25. For example, the projector can project images on any desired plane, object, space, or the like by projection mapping.

The memory 24 is implemented by, for example, a semiconductor memory element, such as a random access memory (RAM) and a flash memory, a hard disk, or an optical disc. The memory 24 stores therein, for example, the X-ray images and the optical images acquired by the X-ray diagnostic apparatus 10, and computer programs for the circuits included in the medical image processing apparatus 20 to implement their functions. The memory 24 may be implemented by a group of servers (cloud) connected to the medical image processing apparatus 20 via the network NW.

The processing circuitry 25 includes an acquisition function 25a and a display control function 25b. The acquisition function 25a is an example of an acquisition unit. The display control function 25b is an example of a display control unit.

For example, the processing circuitry 25 functions as the acquisition function 25a by reading a computer program corresponding to the acquisition function 25a from the memory 24 and executing it. Similarly, the processing circuitry 25 functions as the display control function 25b by reading a computer program corresponding to the display control function 25b from the memory 24 and executing it. The acquisition function 25a and the display control function 25b will be described later in greater detail.

In the medical image processing apparatus 20 illustrated in FIG. 1, each processing function is stored in the memory 24 as a computer-executable program. The processing circuitry 25 is a processor that reads each computer program from the memory 24 and executes it to implement the function corresponding to the computer program. In other words, the processing circuitry 25 that has read a computer program has the function corresponding to the read computer program.

In FIG. 1, a single processing circuitry 25 implements the acquisition function 25a and the display control function 25b. Alternatively, a plurality of independent processors may be combined to constitute the processing circuitry 25, and each processor may execute the computer program to implement the corresponding function. The processing functions of the processing circuitry 25 may be distributed or integrated into a single or a plurality of processing circuits as appropriate.

The processing circuitry 25 may implement the functions using a processor of an external device connected via the network NW. For example, the processing circuitry 25 implements the functions illustrated in FIG. 1 by reading the computer programs corresponding to the respective functions from the memory 24 and executing them, and using a group of servers (cloud) connected to the medical image processing apparatus 20 via the network NW as computing resources.

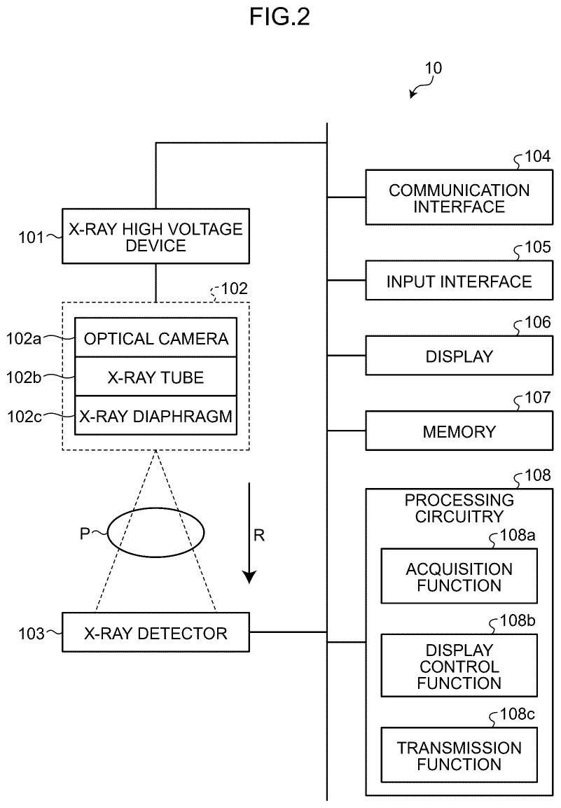

Next, an example of the configuration of the X-ray diagnostic apparatus 10 is described with reference to FIG. 2. FIG. 2 is a block diagram of an example of the configuration of the X-ray diagnostic apparatus 10 according to the first embodiment. As illustrated in FIG. 2, the X-ray diagnostic apparatus 10 includes an X-ray high voltage device 101, an imaging unit 102, and an X-ray detector 103. The imaging unit 102 includes an optical camera 102a, an X-ray tube 102b, and an X-ray diaphragm 102c.

The X-ray high voltage device 101 supplies high voltage to the X-ray tube 102b under the control of processing circuitry 108. For example, the X-ray high voltage device 101 includes an electric circuit, such as a transformer and a rectifier, and includes a high voltage generation device that generates high voltage to be applied to the X-ray tube 102b and an X-ray control device that controls the output voltage according to the X-rays output by the X-ray tube 102b. The high voltage generation device may have a transformer system or an inverter system.

The optical camera 102a is a device that performs optical imaging and acquires optical images. The optical camera 102a is, for example, a digital camera including an image sensor, such as a charge-coupled device (CCD) and a complementary metal-oxide semiconductor (CMOS). While the optical camera 102a typically performs imaging using visible light, it may perform imaging using invisible light, such as infrared light. The optical camera 102a may be capable of capturing only still images or capturing video.

The X-ray tube 102b is a vacuum tube including a cathode (filament) that generates thermoelectrons and an anode (target) that generates X-rays due to the thermoelectrons colliding therewith. The X-ray tube 102b generates X-rays by outputting the thermoelectrons from the cathode to the anode using the high voltage supplied from the X-ray high voltage device 101.

The X-ray diaphragm 102c includes a collimator that narrows the irradiation range of the X-rays generated by the X-ray tube 102b and a filter that adjusts the X-rays output from the X-ray tube 102b.

The collimator in the X-ray diaphragm 102c includes, for example, four slidable aperture blades. The collimator slides the aperture blades to collimate the X-rays generated by the X-ray tube 102b and irradiates a subject P with the collimated X-rays. The aperture blades are plate-like members made of lead or other material and are provided near the X-ray irradiation port of the X-ray tube 102b to adjust the irradiation range of X-rays.

To reduce the radiation dose to the subject P and improve the image quality of the X-ray image, the filter in the X-ray diaphragm 102c changes the quality of the X-rays transmitted therethrough depending on its material and thickness. Thus, the filter reduces the soft X-ray components easily absorbed by the subject P and reduces the high-energy components that cause reduced contrast in the X-ray image. The filter changes the dose and the irradiation range of the X-rays depending on its material, thickness, position, and other factors and attenuates the X-rays output from the X-ray tube 102b to the subject P such that the X-rays have a predetermined distribution.

For example, the X-ray diaphragm 102c includes a drive mechanism, such as a motor and actuator, and operates the drive mechanism under the control of the processing circuitry 108, which will be described later, thereby controlling X-ray irradiation. The X-ray diaphragm 102c, for example, applies a drive voltage to the drive mechanism due to control signals received from the processing circuitry 108 and adjusts the aperture of the aperture blades of the collimator, thereby controlling the irradiation range of X-rays with which the subject P is irradiated. The X-ray diaphragm 102c, for example, applies a drive voltage to the drive mechanism due to control signals received from the processing circuitry 108 and adjusts the position of the filter, thereby controlling the distribution of the dose of X-rays with which the subject P is irradiated.

The X-ray detector 103 is, for example, an X-ray flat panel detector (FPD) including detection elements arrayed in a matrix. The X-ray detector 103 detects X-rays output from the X-ray tube 102b and transmitted through the subject P and outputs detection signals corresponding to the detected X-ray dose to the processing circuitry 108. The X-ray detector 103 may be an indirect conversion detector including a grid, a scintillator array, and a photosensor array or a direct conversion detector including semiconductor elements that convert incident X-rays into electrical signals.

In FIG. 2, the imaging direction of the X-ray image is illustrated as an X-ray irradiation direction R. The X-ray irradiation direction R is a direction in which the X-rays output from the X-ray tube 102b travel. Strictly speaking, the X-ray beam is likely to have a cone shape as illustrated in FIG. 2, and its traveling direction is not constant when focusing on individual X-ray photons. The X-ray irradiation direction R according to the present embodiment is described as one direction that can be represented by one arrow. For example, the X-ray irradiation direction R is a direction along the center line of the spreading cone beam X-rays. Alternatively, for example, the X-ray irradiation direction R is a direction from the X-ray focus to the center of the X-ray detector 103.

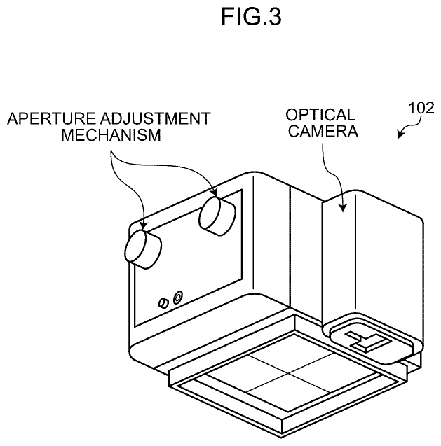

In the present embodiment, the imaging direction of the optical image captured by the optical camera 102a substantially matches the X-ray irradiation direction R. An example of the imaging unit 102 is illustrated in FIG. 3. FIG. 3 illustrates the appearance of the imaging unit 102, and the X-ray diaphragm 102c is housed in the housing. The X-rays generated in the X-ray tube 102b are output to the outside through the X-ray irradiation port and the aperture adjustment mechanism of the X-ray diaphragm 102c. The optical camera 102a is provided on the side surface of the housing that houses the X-ray diaphragm 102c. In the case illustrated in FIG. 3, the X-ray images and the optical images can be acquired in a common imaging direction, although errors occur depending on the distance between the aperture adjustment mechanism and the optical camera 102a.

A targeted body part for the X-ray images and the optical images is not particularly limited. For example, the X-ray images and the optical images may be acquired for the whole body of the subject P or part of it. The posture of the subject P at the time of imaging is not limited, and the X-ray images and the optical images can be acquired for the subject P in any desired posture, such as lying, standing, and seated positions. The imaging unit 102 and the X-ray detector 103 may be held by different holding mechanisms or integrally held by a C-arm or the like. The X-ray diagnostic apparatus 10 may be a portable apparatus, and the imaging unit 102 and the X-ray detector 103 may be manually installed by a user, such as a doctor.

In other words, the type of the X-ray diagnostic apparatus 10 is not limited, and the X-ray diagnostic apparatus 10 can be used for any desired X-ray examination, such as plain X-ray examination, X-ray angiography, X-ray TV, and general X-ray imaging. Depending on the type, the X-ray diagnostic apparatus 10 can include additional components, such as a couchtop for placing the subject P and a C-arm for holding the imaging unit 102 and the X-ray detector 103 as appropriate.

The X-ray diagnostic apparatus 10 further includes a communication interface 104, an input interface 105, a display 106, a memory 107, and a processing circuitry 108. The communication interface 104, the input interface 105, the display 106, and the memory 107 can be configured in the same manner as the communication interface 21, the input interface 22, the display 23, and the memory 24 illustrated in FIG. 1, so explanation thereof is omitted.

The processing circuitry 108 includes an acquisition function 108a, a display control function 108b, and a transmission function 108c. The acquisition function 108a is an example of a acquisition unit. The display control function 108b is an example of a display control unit. For example, the processing circuitry 108 functions as the acquisition function 108a by reading a computer program corresponding to the acquisition function 108a from the memory 24 and executing it. Similarly, the processing circuitry 108 functions as the display control function 108b by reading a computer program corresponding to the display control function 108b from the memory 24 and executing it. Similarly, the processing circuitry 108 functions as the transmission function 108c by reading a computer program corresponding to the transmission function 108c from the memory 24 and executing it.

The acquisition function 108a, for example, controls the X-ray high voltage device 101 and adjusts the voltage supplied to the X-ray tube 102b to control the X-ray dose with which the subject P is irradiated and turning on/off. The acquisition function 108a, for example, controls the operation of the X-ray diaphragm 102c and adjusts the aperture of the aperture blades included in the collimator, thereby controlling the irradiation range of X-rays with which the subject P is irradiated. The acquisition function 108a also controls the operation of the X-ray diaphragm 102c and adjusts the position of the filter, thereby controlling the irradiation range of X-rays and X-ray conditions. If the X-ray diagnostic apparatus 10 includes a couchtop or a C-arm, the acquisition function 108a can control the irradiation range and the irradiation angle of X-rays by controlling their operations. The acquisition function 108a generates an X-ray image based on the detection signals received from the X-ray detector 103 and stores it in the memory 107. The acquisition function 108a may perform various kinds of image processing on the generated X-ray image. For example, the acquisition function 108a performs noise reduction and scattered radiation correction on the X-ray image using an image processing filter.

The acquisition function 108a also controls the optical camera 102a to acquire optical images. For example, the acquisition function 108a controls the imaging conditions, such as the aperture value and the shutter speed in the optical camera 102a. The acquisition function 108a generates an optical image based on the detection signals received from the image sensor included in the optical camera 102a and stores it in the memory 107. The acquisition function 108a may perform various kinds of image processing on the generated optical image.

The display control function 108b controls display on the display 106. The display control function 108b displays, for example, a GUI for receiving various instructions and settings from the operator, the X-ray images and the optical images acquired by the acquisition function 108a, and other data on the display 106. The transmission function 108c transmits various kinds of information via the network NW. For example, the transmission function 108c transmits the X-ray images and the optical images acquired by the acquisition function 108a to the medical image processing apparatus 20 and the image storage apparatus 30.

In the X-ray diagnostic apparatus 10 illustrated in FIG. 2, each processing function is stored in the memory 107 as a computer-executable program. The processing circuitry 108 is a processor that reads each computer program from the memory 107 and executes it to implement the function corresponding to the computer program. In other words, the processing circuitry 108 that has read a computer program has the function corresponding to the read computer program.

In FIG. 2, a single processing circuitry 108 implements the acquisition function 108a, the display control function 108b, and the transmission function 108c. Alternatively, a plurality of independent processors may be combined to constitute the processing circuitry 108, and each processor may execute the computer program to implement the corresponding function. The processing functions of the processing circuitry 108 may be distributed or integrated into a single or a plurality of processing circuits as appropriate.

The processing circuitry 108 may implement the functions using a processor of an external device connected via the network NW. For example, the processing circuitry 108 implements the functions illustrated in FIG. 2 by reading the computer programs corresponding to the respective functions from the memory 107 and executing them and using a group of servers (cloud) connected to the X-ray diagnostic apparatus 10 via the network NW as computing resources.

The term “processor” used in the description above refers to, for example, a central processing unit (CPU), a graphics processing unit (GPU), or a circuit, such as an application specific integrated circuit (ASIC) and a programmable logic device (e.g., a simple programmable logic device (SPLD), a complex programmable logic device (CPLD), and a field programmable gate array (FPGA)). The processor reads and executes the computer programs stored in the memory to implement the functions.

In FIG. 1, a single memory 24 stores therein the computer programs corresponding to the respective processing functions. The embodiments, however, are not limited thereto. For example, a plurality of memories 24 may be distributed, and the processing circuitry 25 may read each computer program from the corresponding individual memory 24. Also in FIG. 2, a plurality of memories 107 may be distributed, and the processing circuitry 108 may read each computer program from the corresponding individual memory 107. Instead of being stored in the memory, the computer programs may be incorporated directly in the circuit of the processor. In this case, the processor reads and executes the computer programs incorporated in the circuit to implement the functions.

The above has described the entire configuration of the medical image processing system 1 including the X-ray diagnostic apparatus 10, the medical image processing apparatus 20, and the image storage apparatus 30. The following describes the processing performed by the medical image processing system 1 in greater detail with reference to the drawings.

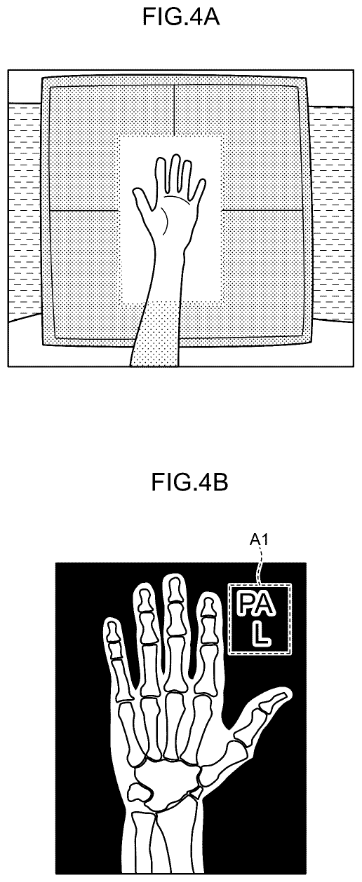

First, the X-ray diagnostic apparatus 10 captures an X-ray image and an optical image. For example, FIG. 4A illustrates the optical image, and FIG. 4B illustrates the X-ray image. In FIGS. 4A and 4B, the “left hand” of the subject P is the targeted body part. The images in FIGS. 4A and 4B are obtained by, for example, imaging the “left hand” of the subject P placed in a supine position on the couchtop. The imaging areas of the X-ray image and the optical image do not necessarily match, and they simply need to include a common targeted body part. For example, the X-ray image may be acquired for the minimum area including the targeted body part, and the optical image may be acquired for a larger area including the targeted body part and its surroundings.

The X-ray image and the optical image share the same imaging direction as described above. For example, the X-ray image and the optical image can be acquired in a common imaging direction by the X-ray tube 102b and the optical camera 102a configured as a unit as illustrated in FIG. 3.

The X-ray tube 102b and the optical camera 102a may be configured as separate components. For example, the optical camera 102a may be held by another arm different from that of the X-ray tube 102b, mounted on the ceiling or wall in the examination room, or configured as a wearable device worn by a doctor or other person. The “common imaging direction” is any direction that can be recognized as the same direction with respect to the targeted body part. If the targeted body part is the “left hand”, and both the X-ray image and the optical image are captured from the “palm” side, for example, the directions are included in the examples of the “common imaging direction” if the values of the imaging angle are slightly different.

The X-ray image and the optical image share the same imaging point in time. The acquisition function 108a, for example, performs synchronized imaging of the X-ray image and the optical image and acquires the X-ray image and the optical image at the same point in time. The acquisition function 108a, for example, captures the optical images at regular time intervals or captures them as video. In this case, the acquisition function 108a can identify an optical image captured at a timing close to the imaging point in time of the X-ray image out of the optical images captured at a plurality of points in time as the optical image that shares the same imaging point in time as the X-ray image.

The acquisition function 108a, for example, records the acquired X-ray image and the acquired optical image in the memory 107 in a manner associated with each other. The acquisition function 108a, for example, records the X-ray image and the optical image in the memory 107 as a data set. The acquisition function 108a records the X-ray image and the optical image in the memory 107 with the information for identifying the corresponding optical image attached to the X-ray image or with the information for identifying the corresponding X-ray image attached to the optical image. The transmission function 108c transmits and registers the X-ray image and the optical image in the image storage apparatus 30 in a manner associated with each other. Associating the X-ray image with the optical image may be performed by the transmission function 108c at the time of transmission or by the image storage apparatus 30 after the transmission.

The acquisition function 25a included in the medical image processing apparatus 20 acquires the X-ray image and the optical image from the image storage apparatus 30 via the network NW. The acquisition function 25a may acquire the X-ray image and the optical image directly from the X-ray diagnostic apparatus 10 not via the image storage apparatus 30.

The display control function 25b displays the X-ray image and the optical image on the display 23. For example, the display control function 25b provides a plurality of display screens on the display 23 to display the optical image illustrated in FIG. 4A and the X-ray image illustrated in FIG. 4B in parallel. The display control function 25b may switch the display between the X-ray image and the optical image in response to a user operation or the like.

The correspondence between the displayed X-ray image and the displayed optical image may be difficult to grasp. For example, the X-ray image is what is called a shadow picture, so it may be inverted (flipped) for interpretation. In other words, the optical image is an image of the subject P viewed from the imaging unit 102 side. The X-ray image is also usually an image of the subject P viewed from the imaging unit 102 side, but when inverted, it is an image of the subject P viewed from the X-ray detector 103 side. In other words, when the X-ray image is inverted, the line-of-sight direction becomes opposite. As a result, the appearances of the optical image and the X-ray image do not match, thereby causing confusion about their correspondence. For example, the X-ray image illustrated in FIG. 4B is inverted (flipped), and its appearance does not match that of the optical image illustrated in FIG. 4A.

Annotation information can be superimposed on the images, and it is conceivable to infer the correspondence between the X-ray image and the optical image based on the annotation information. As indicated by identification information A1 in FIG. 4B, for example, character information, such as “PA” and “L”, may be superimposed on the X-ray image. “PA” stands for “posterior to anterior” and indicates the direction of imaging. “L” stands for “left” and indicates the laterality of the targeted body part. In other words, the character information of “PA” and “L” enables the user to interpret that the X-ray image is obtained by imaging the left hand of the subject P from the back side.

However, interpretation based on the character information is not always accurate. When the targeted body part is the “hand”, for example, interpretations of the character information may vary. For example, the character information of “PA” enables the user to grasp that the image is an X-ray image captured from the back side of the subject P. The user, however, may fail to determine whether the image is acquired by outputting X-rays from the “palm” side to the “back of the hand” side or from the “back of the hand” side to the “palm” side.

In addition, when the character information is manually assigned, human error is expected. When the character information is assigned according to a predetermined algorithm, it is difficult to ensure the accuracy. If the character information is assigned according to pre-registered examination information, for example, the examination information may possibly be incorrectly registered. In addition, a different part from the part scheduled to be captured in the examination information may possibly be captured. For example, if the X-ray images of the “left hand” and the “right hand” of the subject P are scheduled to be captured in sequence, and the X-ray image of the “right hand” is captured first by mistake, the character information of “L (left)” is annotated on the X-ray image of the “right hand”.

Therefore, the medical image processing apparatus 20 superimposes first identification information including figures on the optical image and superimposes second identification information including figures according to the difference in the line-of-sight direction from the optical image on the X-ray image, thereby facilitating grasping the correspondence between the X-ray image and the optical image.

The figure according to the embodiment simply needs to be a figure that can be visually recognized as a certain shape, and its specific shape is not particularly limited. Examples of the figure include, but are not limited to, symbols, characters, etc.

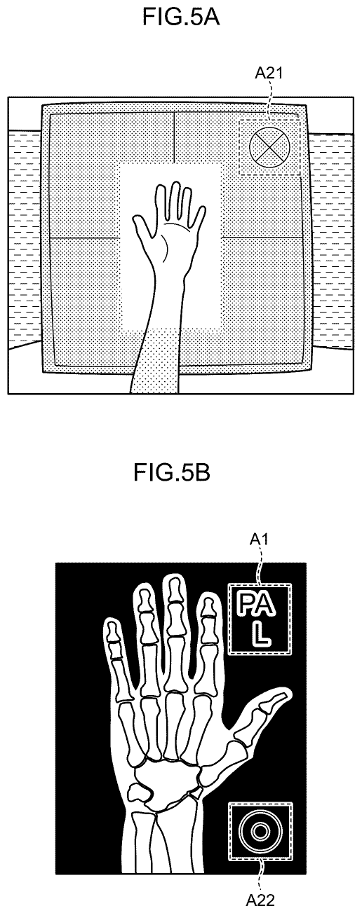

As illustrated in FIG. 5A, for example, the display control function 25b superimposes identification information A21 on the optical image. As illustrated in FIG. 5B, the display control function 25b superimposes identification information A22 on the X-ray image. FIGS. 5A and 5B are schematics of a display example of the identification information according to the first embodiment. The identification information A21 is an example of the first identification information. The identification information A22 is an example of the second identification information.

The optical image in FIG. 5A is an image of the left hand of the subject P viewed from the imaging unit 102 side. In the optical image, the line-of-sight direction matches the imaging direction and is a direction from the front side to the back side with respect to the X-ray tube 102b. Therefore, the display control function 25b displays the identification information A21 including a figure indicating the back side on the optical image in FIG. 5A. The line-of-sight direction is a direction in which the user looks at the object with their eyes. For example, the line-of-sight direction in FIG. 5A is a direction in which the left hand of the subject P is observed from the palm side.

By contrast, the X-ray image in FIG. 5B is inverted with respect to the optical image and is an image of the left hand of the subject P viewed from the X-ray detector 103 side. The imaging direction of the X-ray image in FIG. 5B is opposite to the line-of-sight direction and is a direction from the back side to the front side. Therefore, the display control function 25b displays the identification information A22 including a figure indicating the front side on the X-ray image in FIG. 5B. The display control function 25b, for example, determines whether the line-of-sight directions of the X-ray image and the optical image match and displays the identification information A22 based on the determination result. In other words, the identification information A22 is a figure indicating the X-ray irradiation direction R illustrated in FIG. 2 on the X-ray image.

The identification information A21 is recorded in advance in the memory 107 as predetermined data, such as image data and a combination of text data and font information. The display control function 25b reads the predetermined data from the memory 107 as appropriate and superimposes it on the optical image as the identification information A21. The same is applicable to the identification information A22.

With the identification information A21 and A22, it can be readily grasped that the X-ray image is inverted with respect to the optical image. In other words, the display control function 25b superimposes the identification information A21 on the optical image and the identification information A22 on the X-ray image, thereby facilitating grasping the correspondence between the X-ray image and the optical image.

If the X-ray image is not inverted with respect to the optical image, the display control function 25b displays identification information including a figure indicating the back side instead of the identification information A22. In other words, when the line-of-sight directions of the optical image and the X-ray image are the same, the second identification information superimposed on the X-ray image includes the same figure as that of the first identification information superimposed on the optical image. While FIGS. 5A and 5B illustrate known symbols as the figures indicating the back side and the front side, the embodiment is not limited thereto, and any desired figure can be employed.

The determination of whether the line-of-sight directions of the X-ray image and the optical image match can be made, for example, based on incidental information on the X-ray image. In other words, when the X-ray image is subjected to processing, such as inversion, the history of the processing can be recorded as the incidental information on the X-ray image. The display control function 25b determines whether the X-ray image is inverted based on the incidental information, and if so, it can determine that the line-of-sight directions of the X-ray image and the optical image are different. Alternatively, for example, the display control function 25b determines whether the X-ray image is inverted by image processing, and if so, it can determine that the line-of-sight directions of the X-ray image and the optical image are different. The determination of whether the line-of-sight directions of the X-ray image and the optical image match can be made by any desired method, and the specific method is not limited.

As described above, the acquisition function 25a according to the first embodiment acquires the X-ray image and the optical image of the subject P. The display control function 25b superimposes the first identification information including figures on the optical image and superimposes the second identification information including figures according to the difference in the line-of-sight direction from the optical image on the X-ray image. More specifically, the first identification information according to the first embodiment includes a figure indicating the back side, and the second identification information includes a figure indicating the back side or the front side depending on the difference in the line-of-sight direction from the optical image. With this configuration, the medical image processing apparatus 20 can facilitate grasping the correspondence between the X-ray image and the optical image.

When a doctor interprets the X-ray image, for example, the optical image may be displayed as an aid. Displaying the first identification information and the second identification information facilitates the doctor's grasping the link to the X-ray image when observing the optical image, thereby improving the efficiency of interpretation.

The X-ray image and the optical image may be presented not only to the doctor but also to the subject P. When the doctor explains the results of a diagnosis to the subject P, for example, the X-ray image and the optical image may be used as supplementary materials. The subject P is often a person who does not have expertise in the medical field and may have difficulty understanding the X-ray image unfamiliar to the subject P. Displaying the first identification information and the second identification information enables the subject P to observe the X-ray image based on the optical image familiar to the subject P and facilitates their understanding the X-ray image.

The X-ray image and the optical image can be used for purposes other than interpretation. For example, the X-ray image and the optical image can be used for positioning during imaging.

Specifically, X-ray images of the same subject may be repeatedly acquired in routine checkups, postoperative follow-up, or the like. In this case, positioning is performed in each imaging so as to reproduce the imaging direction, imaging position, posture of the subject P, or the like in the previous imaging, and the X-ray image and the optical image can be used as reference information for the positioning.

To perform positioning, for example, the display control function 25b displays the X-ray image and the optical image acquired in the previous examination with the first identification information and the second identification information. If a plurality of examinations have been performed in the past, the display control function 25b can display a plurality of pairs of the X-ray image and the optical image. Healthcare professionals, such as doctors, who perform positioning can select an X-ray image particularly suitable for observation out of the previously acquired X-ray images and reproduce the posture or the like at the time when the X-ray image is acquired referring to the corresponding optical image.

As indicated by the identification information A1 in FIG. 5B, character information indicating the imaging direction and the laterality can be assigned besides the first identification information and the second identification information. The character information and the first and the second identification information are assigned from different perspectives. If one of them is incorrectly assigned, the other can be correctly assigned. In other words, by recording a plurality of kinds of annotation information from different perspectives, errors, if any, can be readily detected, and the reliability of the annotation information can be increased.

The first embodiment has described an example where the identification information including the figure indicating the back side or the front side is displayed. By contrast, a second embodiment describes an example where identification information including an asymmetrical figure is displayed. The medical image processing system 1 according to the second embodiment has the same configuration as the medical image processing system 1 illustrated in FIGS. 1 and 2. In the following description, the points described in the first embodiment are denoted by the same reference numerals, and explanation thereof is omitted.

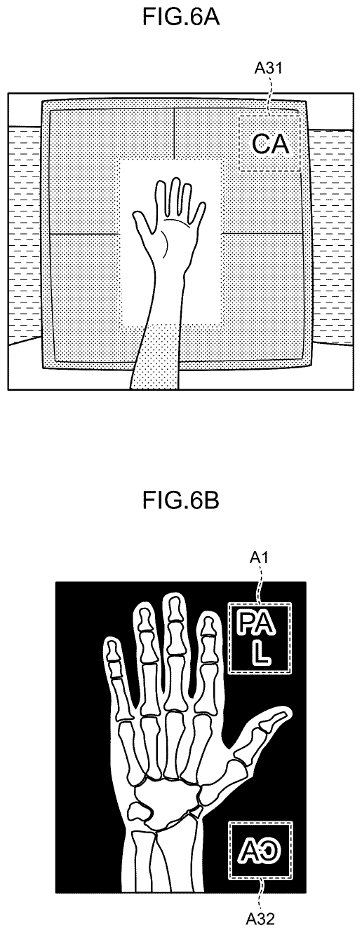

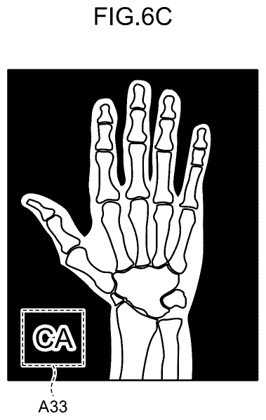

As illustrated in FIG. 6A, for example, the display control function 25b superimposes identification information A31 on the optical image. As illustrated in FIG. 6B, the display control function 25b superimposes identification information A32 on the X-ray image. FIGS. 6A and 6B are schematics of a display example of the identification information according to the second embodiment. The identification information A31 is an example of the first identification information. The identification information A32 is an example of the second identification information.

The optical image in FIG. 6A is an image of the left hand of the subject P viewed from the imaging unit 102 side. By contrast, the X-ray image in FIG. 6B is inverted with respect to the optical image and is an image of the left hand of the subject P viewed from the X-ray detector 103 side. In other words, the line-of-sight directions of the optical image and the X-ray image are different in FIGS. 6A and 6B.

The identification information A31 includes the characters “CA” as an asymmetrical figure. If the asymmetrical figure included in the identification information A31 is characters, it is preferably characters having no anatomical meaning, such as “CA”. In other words, it is preferable to avoid the use of the characters like “PA” indicating the imaging direction and “L” indicating the laterality of the targeted body part.

The identification information A32 includes a figure obtained by inverting the characters “CA”. In other words, the display control function 25b inverts the characters “CA” displayed on the optical image and superimposes them on the X-ray image because the X-ray image is inverted with respect to the optical image. With the identification information A31 and A32, it can be readily grasped that the X-ray image is inverted with respect to the optical image. In other words, the display control function 25b superimposes the identification information A31 on the optical image and the identification information A32 on the X-ray image, thereby facilitating grasping the correspondence between the X-ray image and the optical image.

The identification information A31 is recorded in advance in the memory 107 as predetermined data. The display control function 25b reads the predetermined data from the memory 107 as appropriate and superimposes it on the optical image as the identification information A31. The same is applicable to the identification information A32. Alternatively, the display control function 25b may read the data recorded as the identification information A31 from the memory 107 to perform inversion processing on the data, and superimpose the inverted data on the X-ray image as the identification information A32.

If the X-ray image is not inverted with respect to the optical image, the display control function 25b displays the characters “CA” without inverting them instead of the identification information A32. In other words, when the line-of-sight directions of the optical image and the X-ray image are the same, the second identification information superimposed on the X-ray image includes the same figure as that of the first identification information superimposed on the optical image. While the characters “CA” are laterally and vertically asymmetrical, the asymmetrical figure included in the identification information A31 simply needs to be at least laterally asymmetrical. The asymmetrical figure included in the identification information A31 are not limited to the characters, such as “CA”, and may be symbols or other figures.

The display control function 25b may display the optical image and the X-ray image such that their line-of-sight directions are aligned. For example, the display control function 25b receives an input operation to perform “same direction display” from the user when the optical image illustrated in FIG. 6A and the inverted X-ray image illustrated in FIG. 6B are displayed. In this case, the display control function 25b displays the X-ray image in an inverted manner as illustrated in FIG. 6C such that the X-ray image is in the same line-of-sight direction as the optical image illustrated in FIG. 6A. At this time, the display control function 25b displays identification information A33 including the same figure as that of the identification information A31. The identification information A33 is an example of the second identification information. As a result, it can be readily grasped that the X-ray image is not inverted with respect to the optical image and that these images are in the same line-of-sight direction.

To perform “same direction display” illustrated in FIG. 6C, the identification information A1 indicating the imaging direction and the laterality of the X-ray image is not necessarily displayed. This is because the imaging direction and the laterality of the X-ray image can be readily grasped from the optical image subjected to “same direction display” without requiring annotation.

While the position at which the second identification information is superimposed on the X-ray image is not limited, the second identification information is preferably displayed at a position not overlapping the targeted body part of the subject P. As illustrated in FIG. 6C and other figures, for example, the display control function 25b displays the second identification information at a certain position having a sufficient space to display the identification information in the non-body region outside the body of the subject P. An X-ray image of the chest or abdomen, for example, may possibly have no non-body region. In this case, the display control function 25b can display the second identification information at a certain position other than the part to be examined (e.g., specific organ or bone).

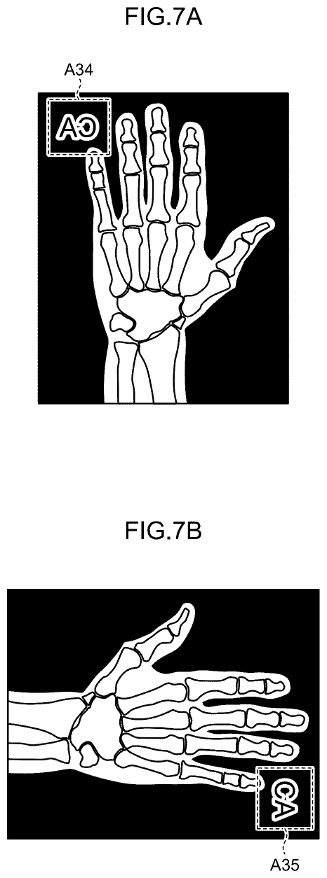

Alternatively, the display control function 25b may display the second identification information in the X-ray image at a position corresponding to the position where the first identification information is displayed on the optical image. For example, the identification information A31 illustrated in FIG. 6A is displayed at a position above the little finger of the left hand of the subject P in the optical image. Therefore, the display control function 25b superimposes identification information A34 at a position above the little finger of the left hand of the subject P in the X-ray image as illustrated in FIG. 7A. This configuration facilitates comparing the identification information A31 with the identification information A34 when the optical image in FIG. 6A and the X-ray image in FIG. 7A are displayed, thereby facilitating grasping that the X-ray image is inverted with respect to the optical image. The identification information A34 is an example of the second identification information.

While the above has described the case where the X-ray image is inverted (flipped), the display mode of the X-ray image can be modified in various manners other than inversion. As illustrated in FIG. 7B, for example, the X-ray image may be rotated with respect to the optical image. More specifically, the X-ray image in FIG. 7B is rotated “90°” clockwise with respect to the optical image illustrated in FIG. 6A. In this case, the display control function 25b superimposes identification information A35 including a figure of the characters “CA” rotated “90°” clockwise on the X-ray image. The identification information A35 is an example of the second identification information. This configuration facilitates grasping that the X-ray image is rotated “90°” clockwise without being inverted when the optical image in FIG. 6A and the X-ray image in FIG. 7B are displayed. To rotate the X-ray image, it is preferable to display the identification information including a laterally and vertically asymmetrical figure, such as the characters “CA”.

Besides the first and the second embodiments described above, other embodiments may be implemented by making various modifications.

For example, the embodiments above have described an example where the X-ray image is inverted (flipped) for interpretation. Inversion of the X-ray image, however, may be performed at a timing other than interpretation, or conversely, it may not be performed at the timing of interpretation. For example, inversion of the X-ray image may be performed at the timing when the X-ray image is generated or stored.

As described above, the acquisition function 108a generates the X-ray image based on the detection signals received from the X-ray detector 103. Let us assume a case where the X-ray detector 103 is an FPD and the detector elements are arrayed in a matrix. When viewing the X-ray detector 103 from the imaging unit 102 in the X-ray irradiation direction R, the leftmost element column is referred to as “E0”, and a plurality of element columns (E0 to En) are arrayed in the horizontal direction. The leftmost pixel column is referred to as “P0”, and an X-ray image composed of a plurality of pixel columns (P0 to Pn) is generated. In this case, the acquisition function 108a determines the pixel values of the leftmost pixel column (P0) in the X-ray image based on the signals detected in the leftmost element column (E0), for example. Similarly, the acquisition function 108a performs the processing on each element column and can determine the pixel values of the pixel columns “P0 to Pn” based on the signals detected in the element columns “E0 to En”, respectively, to generate an X-ray image. In this case, the generated X-ray image is not inverted with respect to the optical image and has the same appearance.

By contrast, the acquisition function 108a can also determine the pixel values of the leftmost pixel column (P0) in the X-ray image based on the signals detected in the rightmost element column (En). The acquisition function 108a performs the processing on each element column and determines the pixel values of the pixel columns “P0 to Pn” based on the signals detected in the element columns “En to E0”, respectively, to generate an X-ray image. In this case, the generated X-ray image is inverted with respect to the optical image. Thus, the X-ray image may be inverted depending on the image generation conditions.

While the above has described an example where the processing is performed column by column, this is given for explanatory purposes only, and the embodiment is not limited by such a processing method. For example, the processing may be performed row by row or not in column or row units. For example, all the signals detected by the detector elements included in the X-ray detector 103 may be processed in parallel. In either case, the X-ray image may be inverted depending on the image generation conditions.

Alternatively, the generated X-ray image may be initially displayed, and the X-ray image may be inverted by the user who refers to the initial display. For example, the user, such as a doctor and a radiological technologist, may operate the X-ray diagnostic apparatus 10 to capture an X-ray image, review the captured X-ray image on the spot, and invert the X-ray image according to the protocols or the like.

Alternatively, the X-ray image may be inverted at the timing of storing it, such as registering the X-ray image in the PACS. To store the X-ray image, for example, it may be converted to digital imaging and communications in medicine (DICOM) and inverted according to the protocols or the like. The user who performs the operation to store the X-ray image may be the same as the user who ordered the imaging or may be another user.

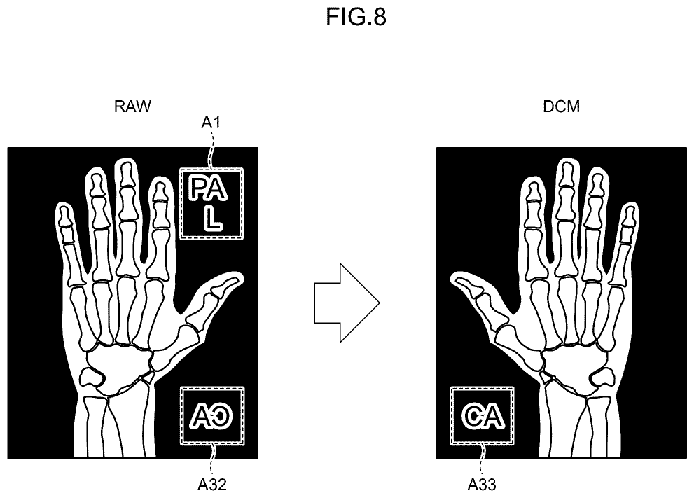

An example where the X-ray image is inverted at the time of storage is illustrated in FIG. 8. In FIG. 8, “DCM” means a DICOM format, and the X-ray image in the DICOM format illustrated on the right is stored. By contrast, “RAW” means raw image data before being converted to the DICOM format. The specific file format of the raw image data is not particularly limited. In the following description, the file format of the raw image data is referred to as a non-DICOM format. In the initial display described above, for example, the X-ray image in the non-DICOM format is displayed. FIG. 8 is a schematic of a display example of the identification information according to a third embodiment.

As in the case described with reference to FIG. 6C, the identification information A33 including the same figure as that of the identification information A31 is displayed with the X-ray image of “DCM” in FIG. 8. As a result, it can be readily grasped that the X-ray image is not inverted with respect to the optical image and that these images are in the same line-of-sight direction.

The X-ray image may be inverted at the time of interpretation. To perform interpretation, for example, the X-ray image registered in the PACS is imported and displayed on a viewer. The viewer is an apparatus that can display medical images managed in a DICOM-compliant format. The viewer may be part of the X-ray diagnostic apparatus 10 or a separate apparatus. The user (radiologist) who performs interpretation can invert the X-ray image as needed while viewing it on the viewer.

As described above, inversion of the X-ray image can be performed under various situations. For example, the X-ray image may be inverted when it is generated according to the image generation conditions or when it is initially displayed, stored, or interpreted. At any of the timings, the display control function 25b can superimpose the first identification information including figures on the optical image and superimpose the second identification information including figures according to the difference in line-of-sight direction from the optical image on the X-ray image as in the first and the second embodiments described above.

For example, the display control function 25b determines whether the line-of-sight directions of the X-ray image and the optical image match and superimposes the second identification information on the X-ray image based on the determination result as in the first and the second embodiments described above. The determination of whether the line-of-sight directions of the X-ray image and the optical image match can be made based on incidental information on the X-ray image, image processing on the X-ray image, or any other desired method.

For example, the incidental information on the X-ray image records information indicating whether inversion of the X-ray image has been performed at a step where the inversion is likely to be inverted. At the timing of interpretation, for example, information is recorded indicating whether inversion has been performed at each step of generation, initial display, and storage of the X-ray image. At the timing of initial display, information is recorded indicating whether inversion has been performed when the X-ray image is generated.

The display control function 25b superimposes the second identification information on the X-ray image based on the information indicating whether inversion has been performed. For example, if the number of times the inversion has been performed is an even number of times (including zero times), the display control function 25b determines that the line-of-sight directions of the X-ray image and the optical image match and displays the identification information A33. By contrast, if the number of times the inversion has been performed is an even number of times, the display control function 25b determines that the line-of-sight directions of the optical image and the X-ray image are different and displays the identification information A32.

The embodiments above have described that the identification information is superimposed on the X-ray image and the optical image. In other words, the identification information according to the embodiments above is digitally assigned. The embodiments, however, are not limited thereto, and the identification information may be physically assigned, thereby acquiring the X-ray image and the optical image with the identification information displayed thereon.

For example, an optical image with the characters “CA” displayed thereon can be acquired by placing an object in the shape of the characters “CA” in the imaging area and performing imaging with the optical camera 102a. An X-ray image with the characters “CA” displayed thereon can be acquired by making the object with radiopaque material, such as lead. When the X-ray image is inverted, the characters “CA” in the image are also inverted. In other words, when the identification information is physically assigned, the correspondence between the X-ray image and the optical image can be readily grasped as in the case illustrated in FIGS. 6A and 6B.

Assigning the identification information digitally has several advantages over assigning it physically. To assign the identification information physically, it is necessary to prepare an object having a predetermined shape and place it in the imaging area. By contrast, when the identification information is superimposed on the X-ray image and the optical image, such an effort is not required, and the workload of the user can be reduced. In addition, the physically assigned identification information may possibly be difficult to see due to its size or color. By contrast, when the identification information is superimposed on the X-ray image and the optical image, the size and color of the identification information can be optionally adjusted for better visibility and can be changed according to the user's preferences.

The embodiments above have described an example where the X-ray image and the optical image are displayed on the medical image processing apparatus 20. The embodiments, however, are not limited thereto. For example, the X-ray diagnostic apparatus 10 may perform the same display as in the medical image processing apparatus 20. Specifically, the acquisition function 108a acquires the X-ray image and the optical image and stores them in the memory 107. The display control function 108b superimposes, on the display 106, the first identification information including figures on the optical image and superimposes the second identification information including figures according to the difference in the line-of-sight direction from the optical image on the X-ray image.

While the optical camera 102a is included in the X-ray diagnostic apparatus 10, they may be configured as separate apparatuses. In other words, the optical camera 102a may acquire the optical images of the subject P under the control of a processing circuit different from the processing circuitry 108.

The components of the apparatuses according to the embodiments described above are functionally conceptual and are not necessarily physically configured as illustrated in the drawings. In other words, the specific forms of distribution and integration of the apparatuses are not limited to those illustrated in the drawings. All or part of the components may be functionally or physically distributed or integrated in desired units depending on various loads, states of use, and the like. All or desired part of the processing functions performed by the apparatuses are implemented by a CPU or a computer program analyzed and executed by the CPU or as hardware by wired logic.

The medical image processing method described in the embodiments above can be performed by executing a computer program prepared in advance on a computer, such as a personal computer and a workstation. This computer program can be distributed via a network, such as the Internet. The computer program is recorded in a non-transitory computer readable medium, such as a hard disk, a flexible disk (FD), a CD-ROM, an MO, and a DVD, and can be executed by being read from the recording medium by a computer.

At least one of the embodiments described above can facilitate grasping the correspondence between an X-ray image and an optical image.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured to:
acquire an X-ray image and an optical image of a subject; and
superimpose first identification information including a figure on the optical image and superimpose second identification information including a figure according to difference in a line-of-sight direction from the optical image on the X-ray image.

2. The medical image processing apparatus according to claim 1, wherein the first identification information includes a figure indicating a back side with respect to an X-ray tube, and the second identification information includes a figure indicating the back side or a front side depending on the difference in the line-of-sight direction from the optical image.

3. The medical image processing apparatus according to claim 1, wherein the first identification information includes an asymmetrical figure, and the second identification information includes a figure obtained by inverting the asymmetrical figure when the line-of-sight directions of the optical image and the X-ray image are different.

4. The medical image processing apparatus according to claim 1, wherein the second identification information includes the same figure as the figure of the first identification information when the line-of-sight directions of the optical image and the X-ray image are the same.

5. The medical image processing apparatus according to claim 1, wherein the processing circuitry is configured to display the X-ray image such that the X-ray image is in the same line-of-sight direction as the optical image, and superimpose the second identification information including the same figure as the figure of the first identification information on the X-ray image.

6. The medical image processing apparatus according to claim 1, wherein the processing circuitry is configured to display the second identification information in the X-ray image at a position corresponding to a position where the first identification information is displayed on the optical image.

7. The medical image processing apparatus according to claim 1, wherein, when the X-ray image is rotated with respect to the optical image, the second identification information includes an asymmetrical figure rotated according to the amount of the rotation.

8. The medical image processing apparatus according to claim 1, wherein the processing circuitry is configured to determine whether the line-of-sight directions of the X-ray image and the optical image match and superimpose the second identification information based on a result of the determination on the X-ray image.

9. The medical image processing apparatus according to claim 1, wherein the figure is a character or a symbol.

10. The medical image processing apparatus according to claim 1, wherein the X-ray image and the optical image include a common targeted body part, share the same imaging direction, and share the same imaging point in time.

11. The medical image processing apparatus according to claim 1, wherein
incidental information on the X-ray image records information indicating whether inversion of the X-ray image has been performed at a step where the inversion is likely to be performed, and
the processing circuitry is configured to determine whether the line-of-sight directions of the optical image and the X-ray image match based on the incidental information and superimpose the second identification information on the X-ray image based on a result of the determination.

12. An X-ray diagnostic apparatus comprising processing circuitry configured to:
acquire an X-ray image of a subject; and
superimpose first identification information including a figure on an optical image of the subject and superimpose second identification information including a figure according to difference in a line-of-sight direction from the optical image on the X-ray image.

13. The X-ray diagnostic apparatus according to claim 12, wherein the first identification information includes a figure indicating a back side with respect to an X-ray tube, and the second identification information includes a figure indicating the back side or a front side depending on the difference in the line-of-sight direction from the optical image.

14. The X-ray diagnostic apparatus according to claim 12, wherein the first identification information includes an asymmetrical figure, and the second identification information includes a figure obtained by inverting the asymmetrical figure when the line-of-sight directions of the optical image and the X-ray image are different.

15. The X-ray diagnostic apparatus according to claim 12, wherein the second identification information includes the same figure as the figure of the first identification information when the line-of-sight directions of the optical image and the X-ray image are the same.

16. The X-ray diagnostic apparatus according to claim 12, wherein the processing circuitry is configured to display the X-ray image such that the X-ray image is in the same line-of-sight direction as the optical image, and superimpose the second identification information including the same figure as the figure of the first identification information on the X-ray image.

17. The X-ray diagnostic apparatus according to claim 12, wherein the processing circuitry is configured to display the second identification information in the X-ray image at a position corresponding to a position where the first identification information is displayed on the optical image.

18. The X-ray diagnostic apparatus according to claim 12, wherein, when the X-ray image is rotated with respect to the optical image, the second identification information includes an asymmetrical figure rotated according to the amount of the rotation.

19. The X-ray diagnostic apparatus according to claim 12, wherein
incidental information on the X-ray image records information indicating whether inversion of the X-ray image has been performed at a step where the inversion is likely to be performed, and
the processing circuitry is configured to determine whether the line-of-sight directions of the optical image and the X-ray image match based on the incidental information and superimpose the second identification information on the X-ray image based on a result of the determination.

20. A non-transitory computer readable medium comprising instructions that cause a computer to execute:
acquiring an X-ray image and an optical image of a subject; and
superimposing first identification information including a figure on the optical image and superimposing second identification information including a figure according to difference in a line-of-sight direction from the optical image on the X-ray image.
